(19) United States
(12) Patent Application Publication
(10) Pub. No.: US 2023/0240752 A1
(43) Pub. Date: Aug. 3, 2023

(54) OPTIMIZING SPINE SCREW PLACEMENT

(71) Applicant: The Cleveland Clinic Foundation, Cleveland, OH (US)

(72) Inventors: David Gurd, Chagrin Falls, OH (US); Kyle Walker, New York, NY (US)

(21) Appl. No.: 18/104,712

(22) Filed: Feb. 1, 2023

Related U.S. Application Data
(60) Provisional application No. 63/305,739, filed on Feb. 2, 2022.

(51) Int. Cl. A61B 34/10

(52) U.S. Cl. CPC A61B 34/10; A61B 2034/105; A61B 2034/104; A61B 2034/107

(57) ABSTRACT

A method for optimization of spine screw placement in a spine of a patient. The method includes a) for a first entry point, defining a first plurality of primary rays; b) eliminating each of the first plurality of primary rays that intersects a boundary of one or more vertebrae of the spine model; c) defining a plurality of parallel rays disposed circumferentially around, and extending parallel to, the associated primary ray at a predetermined radius therefrom; d) iteratively adjusting a length of the plurality of parallel rays associated with each of the first set of optimized screw trajectories until an optimized length is determined; e) presenting a list of the first set of optimized screw trajectories for the first entry point; and f) implanting a spine screw in a vertebra of the patient corresponding to a selected one of the first set of optimized trajectories.

### CROSS-REFERENCE TO RELATED APPLICATIONS

This application claims priority to U.S. Provisional Patent Application No. 63/305,739 filed on Feb. 2, 2022, the entirety of which is incorporated herein by reference.

### TECHNICAL FIELD

The present invention relates generally to the field of surgical planning, and more particularly to automated optimization of spine screw placement.

### BACKGROUND OF THE INVENTION

Surgical planning is a preoperative method of pre-visualizing a surgical intervention, in order to predefine the surgical steps, often in the context of computer assisted surgery. In general, a three-dimensional image of a region of interest of the patient, for example, via magnetic resonance imaging (MRI) or computer tomography (CT), is utilized to plan a surgical intervention within the region of interest.

### BRIEF SUMMARY

There is provided a method for optimization of spine screw placement in a spine of a patient, the method including: a) for a first entry point on a surface of a vertebra among a plurality of vertebrae in a spine model representative of the spine of the patient, defining a first plurality of primary rays respectively representing a plurality of screw trajectories for a spine screw within the model entering from the first entry point; b) eliminating each of the first plurality of primary rays that intersects a boundary of one or more vertebrae of the spine model, representing a surface of an associated vertebra in the patient, thereby establishing a first set of optimized screw trajectories including those of the first plurality of primary rays remaining following this step (b); c) defining, for each of the first set of optimized screw trajectories, a plurality of parallel rays disposed circumferentially around, and extending parallel to, the associated primary ray at a predetermined radius therefrom, and which represent a surface of a spine screw having the screw trajectory represented by the associated primary ray; d) iteratively adjusting a length of the plurality of parallel rays associated with each of the first set of optimized screw trajectories until an optimized length is determined at which the associated plurality of parallel rays present a maximum-length trajectory for a spine screw that does not intersect any the boundary of the one or more vertebra of the spine model; e) presenting a list of the first set of optimized screw trajectories and their associated optimized lengths for the first entry point; and f) implanting a spine screw in a vertebra of the patient corresponding to a selected one of the first set of optimized trajectories.

The foregoing method further including: g) for a second entry point on a surface of a vertebra among the plurality of vertebrae in the spine model, defining a second plurality of primary rays respectively representing a plurality of screw trajectories for a spine screw within the model entering from the second entry point; h) eliminating each of the second plurality of primary rays that intersects a boundary of one or more vertebrae of the spine model, thereby establishing a second set of optimized screw trajectories comprising those of the second plurality of primary rays remaining following this step (h); i) defining, for each of the second set of optimized screw trajectories, a second plurality of parallel rays disposed circumferentially around, and extending parallel to, the associated primary ray at a predetermined radius therefrom, and which represent a surface of a spine screw having the screw trajectory represented by the associated primary ray; j) iteratively adjusting a length of the second plurality of parallel rays associated with each of the second set of optimized screw trajectories until an optimized length is determined at which the associated second plurality of parallel rays present a maximum-length trajectory for a spine screw that does not intersect any of the boundary of the one or more vertebra of the spine model; and k) presenting a list of the second set of optimized screw trajectories and their associated optimized lengths for the second entry point; and l) implanting a spine screw in a vertebra of the patient corresponding to a selected one of the second set of optimized trajectories.

In the foregoing method, the first and second entry points being disposed on a surface of the same vertebra of the plurality of vertebrae.

In the foregoing method, the first and second entry points being disposed on respective surfaces of different vertebrae of the plurality of vertebrae.

In the foregoing method, the spine model including mapping of density of the plurality of vertebrae, wherein the list of the first set of optimized screw trajectories also includes for each optimized screw trajectory thereof a respective first summation of the density of the associated vertebra surrounding or encompassed by the associated plurality of parallel rays, and wherein the list of the second set of optimized screw trajectories also includes for each optimized screw trajectory thereof a respective second summation of the density of the associated vertebra surrounding or encompassed by the associated plurality of parallel rays. The method further including: m) calculating a first respective fixation for each optimized screw trajectory in each of the first and second sets of optimized screw trajectories based on the first or second density summation associated therewith; n) iteratively selecting pairs of the first and second sets of optimized screw trajectories, one from each the set, and calculating an overall fixation for each such pair based on the first respective fixation thereof; and o) presenting a list of the overall fixation and their associated pairs of the first and second sets of optimized screw trajectories.

In the foregoing method, the first respective fixation calculated for each of the first and second sets of optimized screw trajectories is based on a user selected fixation device.

The foregoing method further including: p) calculating a second respective fixation for each of the first and second sets of optimized screw trajectories based on the respective first or second density summation and an alternative fixation device.

In the foregoing method, the first and second entry points being disposed on a surface of the same vertebra of the plurality of vertebrae and the alternative fixation device includes a cross-link connecting a first spline screw in the first entry point to a second spline screw in the second entry point.

In the foregoing method, the spine model is derived via a computed tomography image of the patient's spine.

In the foregoing method, the spine model includes a mapping of density of the plurality of vertebrae.

In the foregoing method, the list of the first set of optimized screw trajectories also includes for each optimized screw trajectory thereof a respective first summation of the density of the associated vertebra encompassed by the associated plurality of parallel rays.

In the foregoing method, the list of the first set of optimized screw trajectories also includes for each optimized screw trajectory thereof a respective first summation of the density of the associated vertebra surrounding the associated plurality of parallel rays.

In the foregoing method, a location of the first entry point is restrained to be within a predetermined distance of the second entry point.

In the foregoing method, the model of the vertebrae including a mapping of density of the plurality of vertebrae, wherein the list of the first set of optimized screw trajectories also includes for each optimized screw trajectory thereof a respective first summation of the density of the associated vertebra surrounding or encompassed by the associated plurality of parallel rays, and wherein the list of the second set of optimized screw trajectories also includes for each optimized screw trajectory thereof a respective second summation of the density of the associated vertebra surrounding or encompassed by the associated plurality of parallel rays. The method further includes: I) calculating a respective pull-out strength for each optimized screw trajectory in each of the first and second sets of optimized screw trajectories based on the first or second density summation associated therewith; m) presenting a list of the pull-out strengths and their associated pairs of the first and second sets of optimized screw trajectories.

In the foregoing method, the pull-out strengths less than a user predetermined value are removed from the list of the pull-out strengths. There is also provided a method for optimization of spine screw placement in a spine of a patient, the method including: a) for a first entry point on a surface of a vertebra among a plurality of vertebrae in a spine model representative of the spine of the patient, defining a first plurality of primary rays respectively representing a plurality of screw trajectories for a spine screw within the model entering from the first entry point; b) eliminating each of the first plurality of primary rays that intersects a boundary of one or more vertebrae of the spine model, representing a surface of an associated vertebra in the patient, thereby establishing a first set of optimized screw trajectories including those of the first plurality of primary rays remaining following this step (b); c) defining, for each of the first set of optimized screw trajectories, a plurality of parallel rays disposed circumferentially around, and extending parallel to, the associated primary ray at a predetermined radius therefrom, and which represent a surface of a spine screw having the screw trajectory represented by the associated primary ray; d) iteratively adjusting a length of the plurality of parallel rays associated with each of the first set of optimized screw trajectories until an optimized length is determined at which the associated plurality of parallel rays present a maximum-length trajectory for a spine screw that does not intersect any the boundary of the one or more vertebra of the spine model; e)) calculating a respective first summation of the density of the associated vertebra surrounding or encompassed by the associated plurality of parallel rays based on a mapping of density of the plurality of vertebrae; f) calculating a respective pull-out strength for each optimized screw trajectory in each of the first set of optimized screw trajectories based on the first density summation associated therewith; g) presenting a list of the first set of optimized screw trajectories and their associated optimized lengths for the first entry point and the pull-out strengths; and and f) implanting a spine screw in a vertebra of the patient corresponding to a selected one of the first set of optimized trajectories.

In the foregoing method, the spine model is derived via a 3-dimensional or volumetric imaging methodology of the patient's spine.

In the foregoing method, the pull-out strengths less than a user predetermined value are removed from the list of the pull-out strengths.

There is also provided, a non-transitory computer readable medium having instructions thereon that, when executed by a computer perform a method for optimization of spine screw placement in a spine of a patient. The method comprising, a) for a first entry point on a surface of a vertebra among a plurality of vertebrae in a spine model representative of the spine of the patient, defining a first plurality of primary rays respectively representing a plurality of screw trajectories for a spine screw within the model entering from the first entry point; b) eliminating each of the first plurality of primary rays that intersects a boundary of one or more vertebrae of the spine model, representing a surface of an associated vertebra in the patient, thereby establishing a first set of optimized screw trajectories including those of the first plurality of primary rays remaining following this step (b); c) defining, for each of the first set of optimized screw trajectories, a plurality of parallel rays disposed circumferentially around, and extending parallel to, the associated primary ray at a predetermined radius therefrom, and which represent a surface of a spine screw having the screw trajectory represented by the associated primary ray; d) iteratively adjusting a length of the plurality of parallel rays associated with each of the first set of optimized screw trajectories until an optimized length is determined at which the associated plurality of parallel rays present a maximum-length trajectory for a spine screw that does not intersect any the boundary of the one or more vertebra of the spine model; and e) presenting a list of the first set of optimized screw trajectories and their associated optimized lengths for the first entry point

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 illustrates a 3D model of an exemplary spine;

FIG. 2A illustrates a cross-section side view showing exemplary spine screws inserted into vertebrae of the spine of a patient;

FIG. 2B illustrates a dorsal view of exemplary spine screws inserted into vertebrae of the spine of a patient;

FIG. 3 illustrates a method for automated optimization of spine screw placement;

FIG. 4 schematically illustrates a system for automated optimization of spine screw placement;

FIG. 5 is a schematic block diagram illustrating an exemplary system of hardware components capable of implementing examples of the systems and methods disclosed herein;

FIG. 6A illustrates a method for automated optimization of spine screw placement;

FIG. 6B illustrates a spherical coordinate system for defining a plurality of rays;

FIG. 6C illustrates a series of generated primary rays defining a plurality of cones, all emanating from a common entry point;

FIG. 6D illustrates a series of eliminated and acceptable primary rays;

FIG. 6E illustrates a series of acceptable parallel rays;

FIG. 7 illustrates a detailed example method for determining an optimal spine screw trajectory into a vertebra at an entry point using a spine model; and

FIG. 8 illustrates additional steps of the method of FIG. 7.

### DETAILED DESCRIPTION

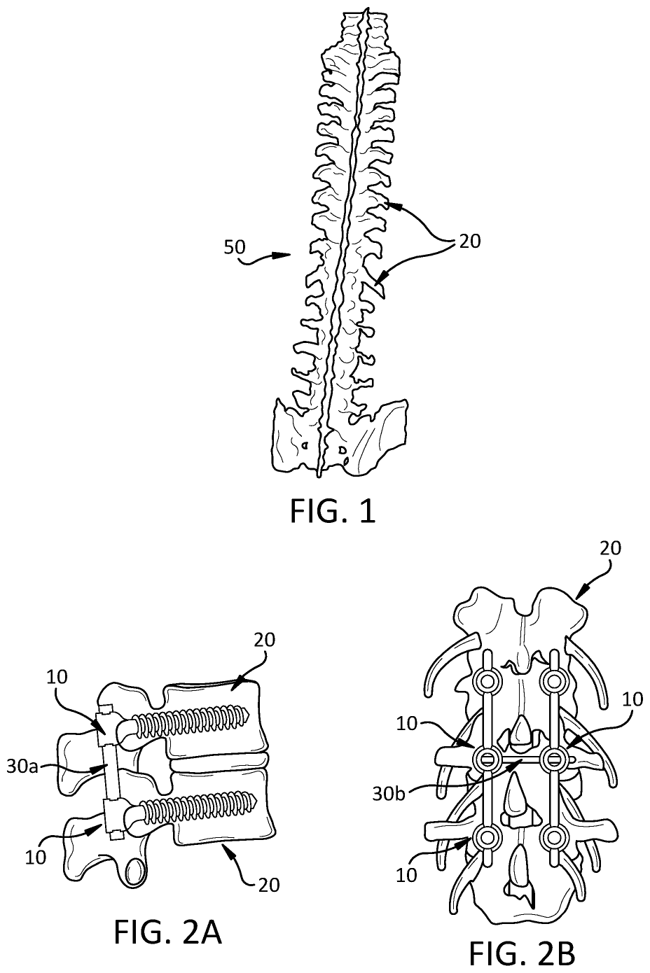

Referring to FIGS. 1-2B, spine screws 10 are used by surgeons for fixation to two or more vertebrae of a spine. FIGS. 1-2B illustrate a spine model 50 comprised of vertebrae 20. The spine screws are used as anchors in order to fix or adjust the relative position or orientation of the vertebrae in order to treat an orthopedic condition, such as scoliosis. The fixation is accomplished by inserting one or more spine screws 10 into each of two or more vertebrae 20. The spine screws 10 in different vertebrae 20 may be fixed to each other using screws and/or rods 30a that can be substantially vertically oriented relative to the longitudinal direction of the spine. It is also contemplated that spine screws 10 in a single vertebra 20 may be fixed to each other via a screw and/or rod 30b that can be substantially laterally oriented relative to the longitudinal direction of the spine. Such vertical screws/rods 30a provide affixation between different vertebrae; whereas such lateral screws/rods 30b provide additional strength and structural reinforcement (e.g. a reinforcing cage) at the level of the subject vertebra. In the embodiment illustrated in FIG. 2A, the vertical screw/rod 30a provides fixation between vertebrae that are directly adjacent one another. It is contemplated that the vertical screws/rods 30a may provide fixation between vertebrae that are not directly adjacent to each other, e.g., the spine screws 10 may be in vertebrae that are spaced one or more vertebrae from each other (not shown).

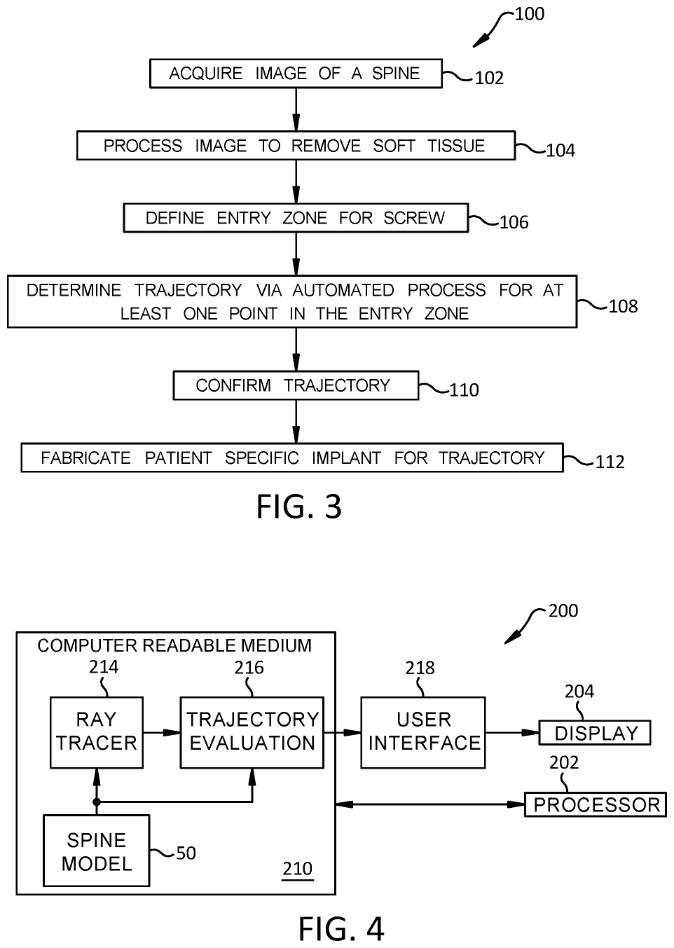

The present application provides systems and methods for planning the insertion of spine screws 10 into vertebrae 20. Referring to FIG. 3, one example of a general method 100 for surgical planning the process of inserting one or more spine screws 10 into the spine is illustrated. The method begins at 102, where an image of a spine is acquired to generate a 3D spine model 50. In one implementation, a computer tomography (CT) image is acquired and used to generate the 3D spine model 50 (FIG. 1). At 104, the image is processed, optionally by a technician, to remove soft tissue from the image, leaving only bony vertebral tissue in the 3D spine model 50 (FIG. 1) with numerous levels of vertebrae 20. At 106, a surgeon defines an entry point or zone for the spine screw 10 on the spine model 50. In general, the entry point or zone will define a point or points on the surface of the vertebra 20 at which the spine screw 10 can be inserted. It is also contemplated that an algorithm may provide a recommended entry point for each vertebra 20, as described in detail below.

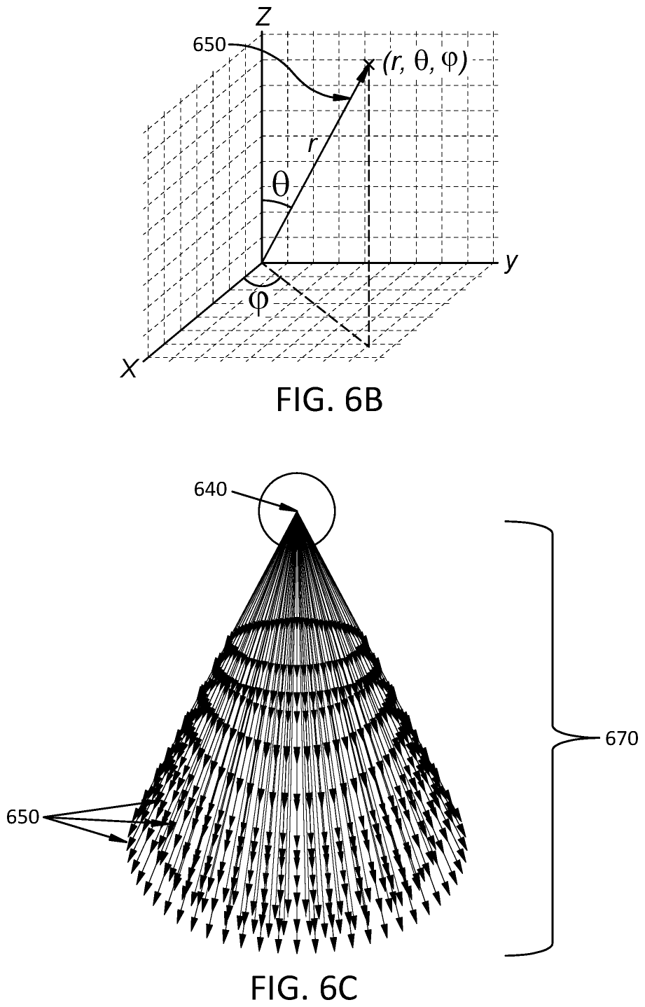

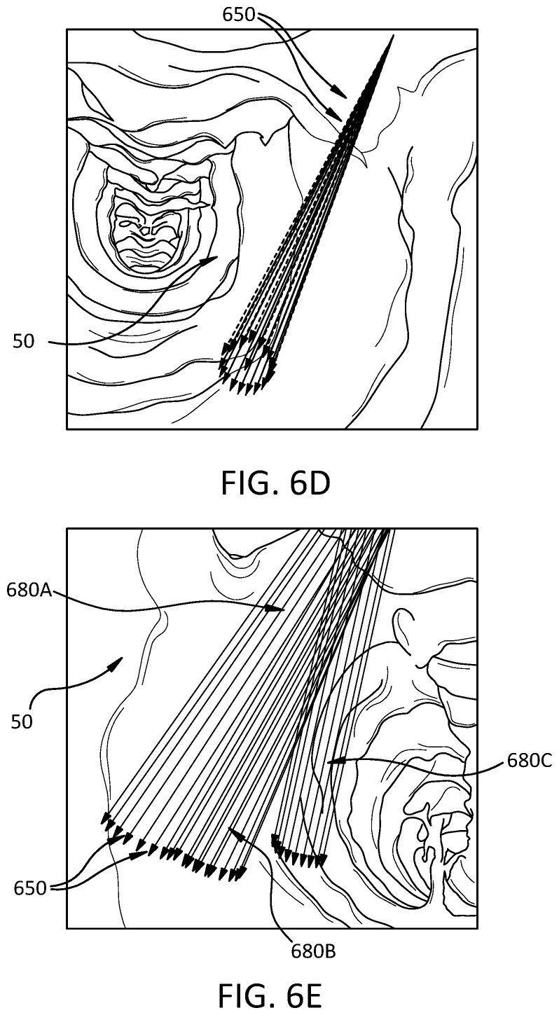

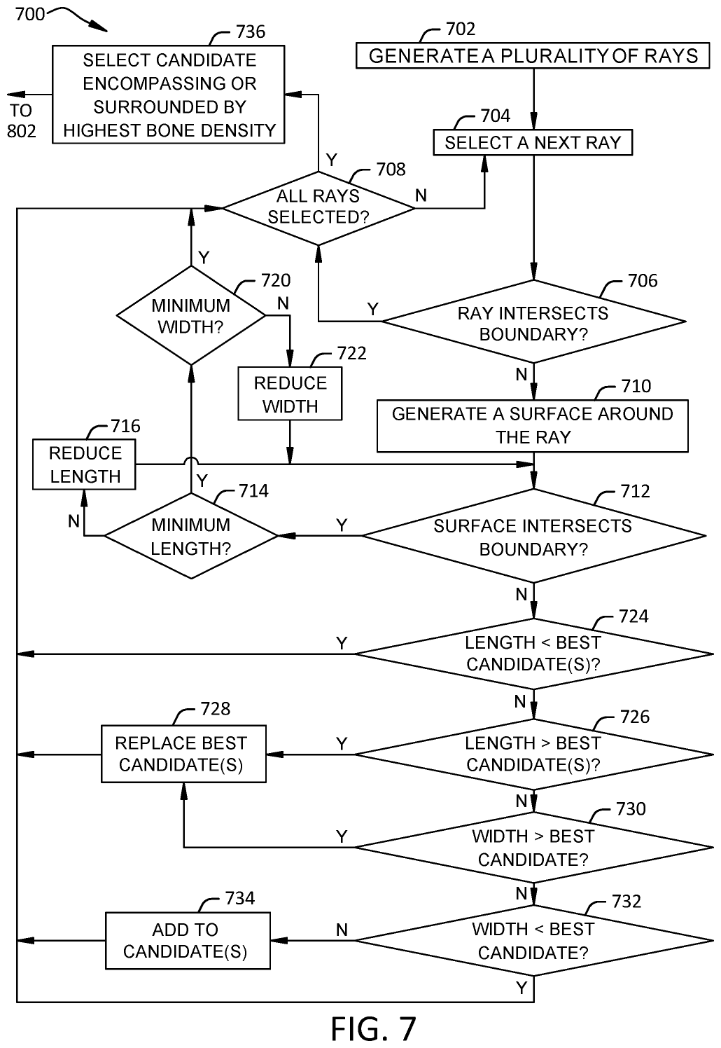

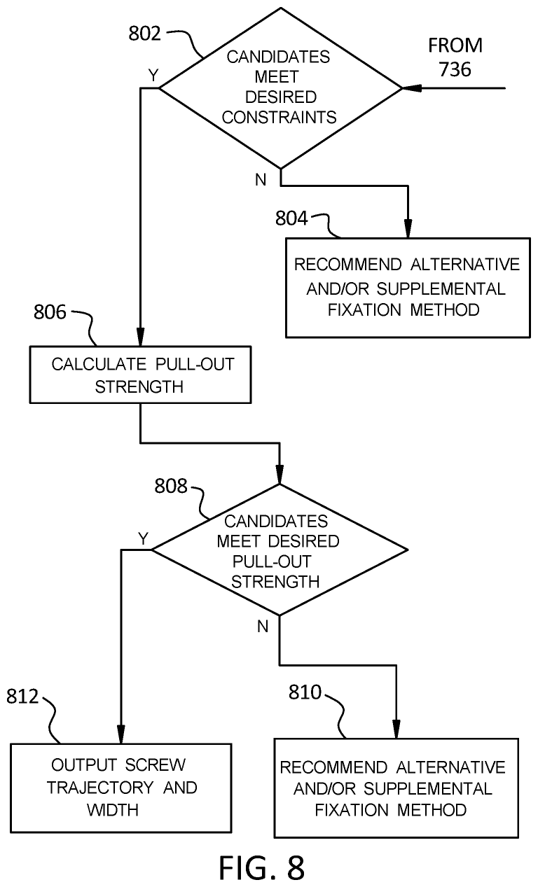

At 108, a trajectory for the spine screw 10 is determined via an automated process at each possible entry point in the defined entry zone. In one example, a ray tracing process (described in detail below) is used to model various trajectories against the vertebral boundaries, and a trajectory is selected to allow for the longest possible spine screw 10 to be inserted. Where multiple trajectories exist that allows for a same length, a trajectory allowing the spine screw 10 of the greatest width is selected. Where multiple trajectories allow for spine screws 10 of the same length and width, the trajectory in the region of highest bone density is selected. An example of such an algorithm is summarized in FIG. 6A and described in further detail in FIGS. 7-8. Returning to FIG. 3 illustrating a general method of surgical planning, at 110, the surgeon confirms the trajectory (which can be ascertained via an algorithm as noted above), and at 112 a patient-specific instrument, configured to affix to the vertebral surface and guide the screw for insertion at the designated entry point and then along the appropriate trajectory, is fabricated. The patient-specific instrument may be a bracket or jig that is manufactured prior to the surgery that orientates the spine screw in the proper trajectory relative to the associated vertebra. It is also contemplated that the trajectory may be used as input into navigation software, a robotic device or other systems to guide the insertion of the spine screw 10 into the vertebra 20.

FIG. 4 illustrates a functional block diagram of a system 200 for automated optimization of the spine screw 10 placement into the vertebra 20. The system 200 includes a processor 202, a non-transitory computer readable medium 210 storing executable instructions that are executable by the processor 202, a display 204 and a user interface 218. The instructions include a three-dimensional spine model 50 having numerous vertebral levels, obtained for example, via computer tomography or another imaging process. As described in detail below, the instructions further include a ray tracer 214 that, for each vertebra in the spine model, generates a set of rays for each of a plurality of potential trajectories for the spine screw 10 into that vertebra of the three-dimensional model from an entry point on a surface thereof. The set of generated rays for each potential trajectory from the entry point includes a first ray representing a center axis of the spine screw 10, and a plurality of parallel rays circumferentially disposed about the first ray and together representing a surface of the spine screw 10. For example, the plurality of parallel rays can be spaced by a common radius from the first ray (corresponding to the center axis of the represented spine screw) to define a cylindrical surface, with the plurality of parallel rays evenly spaced along the cylindrical surface.

The instructions also include a trajectory evaluator 216 that selects at least one trajectory represented by the set of generated rays having a longest length before intersecting a boundary of the subject vertebra in the three-dimensional model. In one implementation, the set of rays associated with each trajectory can be iteratively reduced until no intersection with the boundary is detected. Where multiple rays of similar length are available, one or both of a largest radius of the additional rays or a total bone density encompassed by the cylindrical surface can be used to select a final trajectory.

In the embodiment wherein a total bone density is used by the trajectory evaluator 216, the bone density distribution in the vertebra may be determined using a volumetric density analysis/estimation. In the instance where a surgeon desires to use an awl to create a pilot hole in the vertebra, the volumetric density analysis/estimation may be used to identify the path with the greatest bone density so that use of the awl will further “pack” the bone at the point and along the trajectory where inserted. Packing the bone helps to increases the density of the bone into which the spine screw 10 will thread.

On the other hand, in the instance where a surgeon desires to use a drill to create the pilot hole, the volumetric density analysis/estimation may be used to identify the path which should leave behind higher-density bone around the hole.

The difference lies in the understanding that advancement of an awl compresses (i.e. ‘packs’) bone material surrounding its insertion trajectory, whereas a drill bit removes bone material to excavate the hole. When removing material (with a drill bit), one does not want to remove the densest bone (e.g. via drilling into it). Rather, it would be preferable to drill the pilot hole adjacent to (and not through) the path of densest bone, to ensure strongest possible screw-engagement along the pilot-hole trajectory.

Consequently, the algorithm can select a pilot-hole trajectory through the densest bone material if an awl is to be used to generate that hole, and it can select a trajectory adjacent to (but not through) the densest material if a drill is to be used. In each instance, the algorithm ensures that the bone remaining in the vicinity of the pilot hole will provide the highest possible density for screw-threading engagement.

It will be appreciated that other metrics can be used for selecting a trajectory, including selecting a surface that has a maximum encompassed bone density regardless of the length or width of the screw. The user interface 218 may provide the selected at least one trajectory to a user at the associated display 204.

In one implementation, additional constraints can be applied in selecting the trajectory. Specifically, using finite element analysis, the maximum distance two spine screws 10 can be deviated from one another based on their superior/inferior (or cephalocaudal) distance from one another can be determined. With more than two screws this essentially creates a spline constraint to optimize the screw trajectory at each level (or vertebra) while allowing the spine screws 10 to still be connected by a rod with minimal intrinsic static forces within the system.

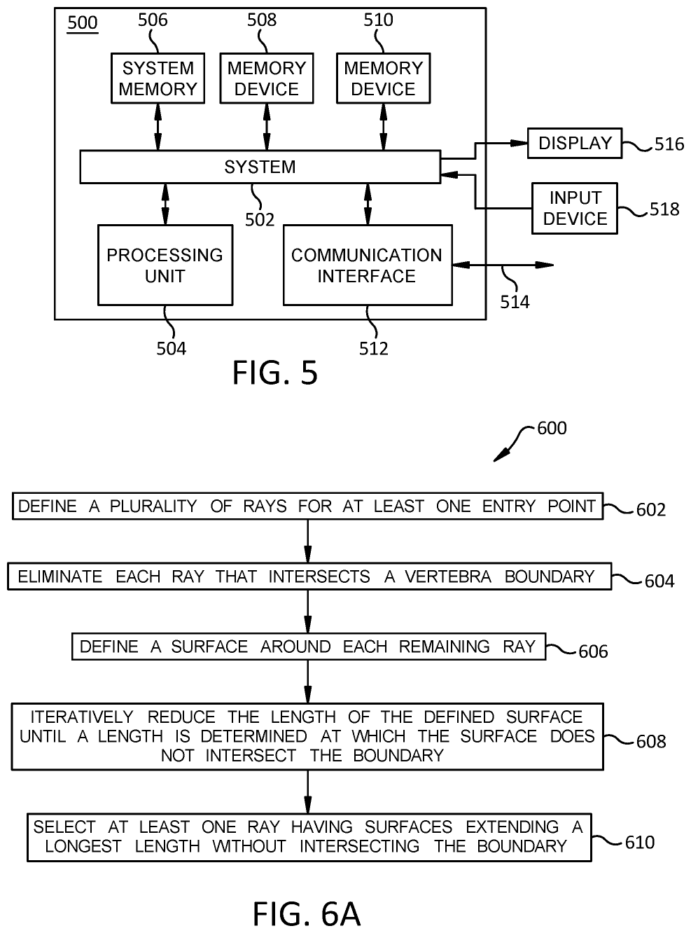

FIG. 5 is a schematic block diagram illustrating a system 500 of hardware components capable of implementing the methods disclosed in detail herein. The system 500 can include various systems and subsystems. The system 500 can be a personal computer, a laptop computer, a workstation, a computer system, an appliance, an application-specific integrated circuit (ASIC), a server, a server blade center, a server farm, etc.

The system 500 can include a system bus 502, a processing unit 504, a system memory 506, memory devices 508 and 510, a communication interface 512 (e.g., a network interface), a communication link 514, a display 516 (e.g., a video screen), and an input device 518 (e.g., a keyboard and/or a mouse). The system bus 502 can be in communication with the processing unit 504 and the system memory 506. The additional memory devices 508 and 510, such as a hard disk drive, server, stand-alone database, or other non-volatile memory, can also be in communication with the system bus 502. The system bus 502 interconnects the processing unit 504, the memory devices 506-510, the communication interface 512, the display 516, and the input device 518. In some examples, the system bus 502 also interconnects an additional port (not shown), such as a universal serial bus (USB) port.

The processing unit 504 can be a computing device and can include an application-specific integrated circuit (ASIC). The processing unit 504 executes a set of instructions to implement the operations of examples disclosed herein. The processing unit can include a processing core.

The additional memory devices 506, 508 and 510 can store data, programs, instructions, database queries in text or compiled form, and any other information that can be needed to operate a computer. The memories 506, 508 and 510 can be implemented as computer-readable media (integrated or removable) such as a memory card, disk drive, compact disk (CD), or server accessible over a network. In certain examples, the memories 506, 508 and 510 can comprise text, images, video, and/or audio, portions of which can be available in formats comprehensible to human beings. Additionally or alternatively, the system 500 can access an external data source or query source through the communication interface 512, which can communicate with the system bus 502 and the communication link 514.

In operation, the system 500 can be used to implement one or more parts of a surgical planning process in accordance with the present invention. Computer executable logic for implementing the surgical planning process resides on one or more of the system memory 506, and the memory devices 508, 510 in accordance with certain examples. The processing unit 504 executes one or more computer executable instructions originating from the system memory 506 and the memory devices 508 and 510. The term “computer readable medium” as used herein refers to any medium that participates in providing instructions to the processing unit 504 for execution, and it will be appreciated that a computer readable medium can include multiple computer readable media each operatively connected to the processing unit.

In view of the structural and functional features described above, an example algorithm in accordance with various aspects of the present invention will be better appreciated with reference to FIGS. 6A-8. While, for purposes of simplicity of explanation, the method and algorithm of FIGS. 6A-8 are shown and described as executing serially, it is to be understood and appreciated that the present invention is not limited by the illustrated order, as some aspects could, in accordance with the present invention, occur in different orders and/or concurrently with other aspects from that shown and described herein.

FIG. 6A illustrates a method 600 for automated optimization of spine screw placement. At 602, for an entry point 640 (FIG. 6C), a plurality of primary rays 650 (FIG. 6C) are defined. Each primary ray 650 represents an axis of a potential spine screw 10 and a potential trajectory of that spine screw 10 in a vertebra within the spine model 212 from the entry point 640 (FIG. 6C). It will be appreciated that the entry point 640 of the rays 650 can be an entry point for the spine screw 10 defined by a surgeon or as recommended by the algorithm (described in detail above). It is contemplated that the surgeon may select more than one entry point 640 (FIG. 6C) for each vertebra. Accordingly, the method 600 may be repeated for each entry point 640 (FIG. 6C) selected by the surgeon.

Referring to FIGS. 6B and 6C, it is contemplated that the plurality of primary rays 650 may be generated to form a series of concentric cones 670 with aperture angles θ. Each cone 670 has a vertex at the entry point 640. The aperture angle θ (FIG. 6B) for the cones may range from a minimum of about 0 degrees to a maximum of about 30 degrees. It is contemplated that the maximum aperture angle θ may be greater than 30 degrees. The plurality of primary rays 650 in each cone 670 may have an angle φ (FIG. 6B) that ranges from about 0 degrees to about 360 degrees in increments of about 5 degrees.

At 604, each of the plurality of primary rays 650 (FIG. 6C) that intersects a boundary of the vertebra 20 in the model is eliminated. Referring to FIG. 6D, an exemplary spine model 50 is illustrated and exemplary primary rays 650 that are eliminated are drawn with dashed lines and exemplary primary rays 650 that are not eliminated are drawn with solid lines.

At 606, for each of the primary rays 650 that is not eliminated, a surface of a spine screw having the screw trajectory associated with the primary ray 650 (FIG. 6C) is defined. In one implementation, the surface is defined as a plurality of evenly spaced parallel rays defining a cylindrical surface around (and parallel to) the primary ray 650 (FIG. 6C) at a preselected radius. FIG. 6E illustrates an exemplary spine model 50 with three surfaces 680A, 680B, 680C defined around three primary rays (not shown because they would be at the center of the respective cylindrical surfaces), all emanating from a common entry point. The cylindrical surfaces 680A, 680B, 680C may be selected to correspond to an outer surface of the largest diameter of the spine screw 10.

At 608, the length of the defined surface is iteratively reduced until a length is determined at which the defined surface 680A, 680B, 680C does not intersect the boundary of the vertebra model. It will be appreciated that the length can be reduced by a constant amount each time or reduced to a next longest length of spine screw 10 available for the procedure. At 610, a set of at least one primary rays having a longest determined length is selected. An optimized trajectory for the spine screw placement can be determined as a trajectory represented by one of the set of at least one primary ray. In one implementation, where multiple surfaces 680A, 680B, 680C extend to a same length, a spine screw 10 having a widest surface and/or encompassing the most total bone density in the vertebra model, as described above, can be selected.

In the embodiment described above, the primary rays were selected by starting with the longest permissible screw and then adding rays around that primary ray to define the surface of the spine screw 10. It is contemplated that the surface of the spine screw 10 can be added before determining the longest permissible screw. In this alternative embodiment, the algorithm may determine that largest diameter of the spine screw 10 that can be used, regardless of the length of the spine screw 10. This alternative embodiment finds particular application where the surgeon prefers a larger diameter screw rather than a longer screw.

FIG. 7 illustrates flow chart representing one example of an algorithm 700 that is used in the method 600 (FIG. 6A) for determining an optimal spine screw trajectory into a vertebra at an entry point using a spine model. The algorithm 700 begins at 702, where the plurality of primary rays 650 (FIG. 6C) are generated from the entry point 640 (FIG. 6C). Each primary ray 650 (FIG. 6C) begins at the entry point 640 (FIG. 6C) and extends for a predetermined length in a selected direction. It will be appreciated that that predetermined length can be equal to a maximum length of the spine screw 10 (FIG. 2A) that might be used in a surgical procedure. The primary rays 650 (FIG. 6C) can be arranged to cover a two-dimensional “grid” of angular values in polar coordinates (see FIG. 6A). In one implementation, the primary rays form the cone 670 (FIG. 6C) with an aperture of approximately thirty degrees, with the individual primary rays 650 (FIG. 6C) separated by approximately five degrees in each direction. This provides approximately four hundred forty total primary rays. The cone 670 (FIG. 6C) can be centered on an axis normal to the surface of the vertebra or on an initial trajectory selected by a surgeon.

At 704, a next primary ray 650 is selected. It will be appreciated that, in the first iteration of the algorithm 700, the “next” primary ray will be a first selected primary ray 650. At 706, it is determined if the selected primary ray 650 intersects a boundary of the vertebrae 20 of the spine model 50. If an intersection is determined (Y), it is assumed that the spine screw 10 will perforate the vertebra 20 if inserted at the trajectory represented by the selected primary ray 650. Accordingly, the trajectory represented by the selected primary ray 650 is rejected, and the algorithm 700 advances to 708, where it is determined if all primary rays 650 have been selected. If not (N), a next primary ray 650 is selected at 704. If no intersection is determined (N), the algorithm 700 advances to 710, where a surface (see, e.g. 680A, 680B, 680C in FIG. 6E) is generated around the selected primary ray 650. In the illustrated algorithm 700, the surface is generated as a plurality of parallel rays evenly spaced in a circle around the selected primary ray 650, with the parallel rays each running parallel to and being spaced from the selected primary ray 650 by a predetermined radius equal to approximately half of a maximum width of the spine screw 10 that might be used in the procedure. Accordingly, the distance between two opposing parallel rays (relative to the selected primary ray 650 equidistant between them) should be equal to a maximum width of the spine screw 10.

At 712, it is determined if the generated surface intersects the vertebra boundary. If so (Y), the algorithm 700 advances to 714, where it is determined if the selected primary ray 650 is at a minimum length, that is, a length approximately equal to that of a shortest spine screw 10 that might be used in the procedure. If the minimum length has not been reached (N), the algorithm 700 advances to 716, where the length of the selected primary ray 650, as well as the parallel rays forming the surface surrounding it, are reduced. This reduction can be by a predetermined amount or by an amount necessary to reduce the length to that of a next shortest spine screw 10 that is available for the procedure. The algorithm 700 then returns to 712.

Returning to 714, if the minimum length has been reached (Y), the algorithm 700 advances to 720, where it is determined if the surface surrounding the selected primary ray 650 is at a minimum width, that is, a width approximately equal to that of a smallest diameter of the spine screw 10 that might be used in the procedure. If so (Y), the trajectory represented by the selected primary ray is rejected, and the algorithm 700 returns to 708, where it is determined if all primary rays have been selected. If not (N), the algorithm 700 advances to 722 where the width of the surface is reduced. This reduction can be by a predetermined amount or by an amount necessary to reduce the width to that of the spine screw 10 of a lower diameter that is available for the procedure. The algorithm 700 then returns to 712.

Returning to 712, if it is determined that the generated surface does not intersect the vertebra boundary (N), the algorithm 700 advances to 724, where it is determined if the length of the selected primary ray 650 is shorter than the current best candidates. Where no best candidate has been selected, this decision defaults to no. If the selected primary ray 650 is shorter than any selected best candidates (Y), the trajectory represented by the selected primary ray is rejected, and the algorithm 700 returns to 708, where it is determined if all primary rays have been selected. If it is determined that the selected primary ray 650 is not shorter than the current best candidates (N), the method advances to 726, where it is determined if the length of the selected primary ray 650 is longer than the current best candidates. Where no best candidate has been selected, this decision defaults to yes. If the selected primary ray is longer than any selected best candidates (Y), all of the best candidates are removed and replaced with the selected primary ray at 728. The algorithm 700 then returns to 708 to determine if all of the primary rays have been selected.

If the selected primary ray is not longer than any selected best candidates (N), it can be presumed that it is of equal length to the best candidates. The algorithm 700 advances to 730, where it is determined if the width of the surface associated with the selected primary ray 650 is greater than that of the current best candidates. If the selected primary ray has an associated surface (composed of the associated circumferentially disposed parallel rays) with a width greater than any selected best candidates (Y), all of the best candidates are removed and replaced with the selected primary ray at 728 and the algorithm 700 returns to 708 to determine if all of the primary rays have been selected. If the selected primary ray does not have an associated surface with a width greater than any selected best candidates (N), it is determined if the width of the surface associated with the selected primary ray is less than that of the current best candidates at 732. If the selected primary ray has a surface with a width less than that of any selected best candidates (Y), the trajectory represented by the selected primary ray is rejected, and the algorithm 700 returns to 708, where it is determined if all primary rays have been selected. If the selected primary ray does not have an associated surface with a width less than any selected best candidates (N), it can presumed that the surface associated with he selected primary ray is of equal length and width to the best candidates. It is thus added to the list of best candidates, without removing any existing candidates at 734. The algorithm 700 then returns to 708, where it is determined if all primary rays have been selected.

Returning to 708, if it is determined that not all of the primary rays have been selected (N), the algorithm 700 returns to 704 to select a next primary ray for evaluation. Once all of the primary rays have been selected for evaluation (Y), the algorithm 700 advances to 736, where a candidate primary ray (and its associated surface composed of the surrounding parallel arrays) encompassing a highest total bone density is selected. Where there is a single best candidate, that candidate can be selected without further evaluation. Where multiple candidates have been identified, however, the bone density within the region encompassed by the associated surface around the selected ray can be summed using the spine model. The candidate primary ray (and associated surface) having the highest value can be selected as the trajectory for the insertion of the spine screw 10.

In the alternative, as described above, the algorithm 700 may be configured so that instead of using the candidate with the highest encompassed bone density the algorithm 700 may select the candidate that will result in the highest bone density surrounding the selected spine screw 10 once installed along the primary-ray trajectory. That will allow the spine screw 10 to thread into the strongest part of the vertebra.

Referring to FIG. 8, in 802, the set of candidates are compared to the desired constraints provided by the surgeon. If the highest encompassed or surrounding bone density does not meet the surgeon's desired value, in 804 the algorithm 700 will recommend alternative or supplemental fixation devices. For example, the algorithm 700 may have received as input from the surgeon the fixation device that the surgeon wishes to use at a given vertebra. Based on the volumetric density analysis/estimation, the algorithm 700 may recommend a different fixation device, e.g. mono-axial, poly-axial, hook, etc., and indicate to what degree the overall fixation can be improved by using the fixation device suggested by the algorithm 700 at the specified location/vertebra.

It is further contemplated that the algorithm 700 may be configured to recommend vertebrae where cross-links, i.e., fixation between spine screws in the same vertebra via lateral rods/screws 30b (see, FIG. 2B) should be used to improve the overall fixation for the patient. It is also contemplated that at 802 the algorithm 700 may be configured to use the results of the volumetric density analysis/estimation to suggest an alternative or supplemental fixation device, e.g. mono-axial, poly-axial, hook, etc., that should be used in each level or vertebra to achieve multi-level planning; i.e. to plan spine screw placement among all of, or even just the most optimized, vertebrae in the vicinity of the portion of the spine in need of therapy. In this manner, in conjunction with determining the suitable fixation device to use at each level, the algorithm 700 may determine the optimal placement of the fixation devices among multiple vertebrae to be constrained together. A different fixation device at each vertebra may be selected to provide optimal overall fixation for the patient, e.g. based on the relative densities/porosities and differing geometric configurations of the different vertebrae. The overall fixation may be based on respective fixation of the associated spine screws 10. The algorithm 700 may determine that although a particular fixation device at one vertebra provides optimal fixation for that vertebra, a different fixation device will provide optimal fixation at a different vertebra, resulting in overall optimized fixation for the patient.

Additionally, the algorithm 700 may be configured to output dimensions for the rods 30a (FIG. 2A) to be used to constrain the levels or vertebrae 20 together, to achieve a desired spinal curvature for the patient. These rods 30a (FIG. 2A) may be manufactured in advance via machining, thereby eliminating the introduction of fatigue therein that would occur if formed by bending during the surgery. Such a pre-formed, machined rod 30a (FIG. 2A) also can be custom tailored to the patient's unique physiology and desired post-procedure spinal geometry, as an output of the algorithm 700 according to the associated multi-level plan for that patient. Such preformed rods 30a (FIG. 2A) also aid in reducing the overall length of the surgery, thereby freeing the operating room for another patient and reducing the amount of time in surgery.

It is also contemplated that the surgeon may use the algorithm to create a virtual custom rod 30a (FIG. 2A) that he has determined will be optimal for the patient. Based on this virtual custom rod, the algorithm may determine the proper placement, length and orientation of the spine screws (or other fixation devices) that will attach to that custom rod.

In 806, the algorithm 700 may use the outputted screw trajectories and widths to calculate predicted pull-out strength. The algorithm 700 may be configured to use historical data regarding pull-out strength for each vertebra 20 (FIG. 2A) to predict the pull-out strength for each vertebra 20 of a given patient. The algorithm 700 may be configured to adjust the calculated pull-out strength based on various factors, including but not limited to, the age of the patient, actual bone density determined by volumetric density analysis/estimation, osteoporotic characteristics, etc. Once the algorithm 700 calculates a predicted pull-out strength for the spine screw in a given vertebra, in 808 the algorithm 700 may then determine if the calculated pull-out strength is greater than or equal to the desired pull-out strength for that vertebra. If the calculated pull-out strength is low, in 810 the algorithm may recommend an alternative and/or supplemental fixation device for increased strength. It is also contemplated that the algorithm 700 may determine that the desired pull-out strength cannot be achieved for the given vertebra. In this instance, the algorithm may recommend placing spine screws in other vertebrae to achieve the desired overall strength and curvature. It is contemplated that the other vertebrae may not necessarily be directly adjacent the given vertebra and may be spaced one or more vertebrae away from the given vertebra.

In real-world situations, a spine screw is unlikely to be pulled out (along its axis) of the patient's vertebra. But pull-out strength can be used as a surrogate to predict more likely real-world biomechanical-load failures, which typically will be cantilever-failures, and not axial ones. For example, pull-out strength will be a function of bone density in the vicinity of the spine screw, the spine screw's size, its length, and the level (vertebra) at which it has been affixed. Using historical data relating pull-out strength as a function of these (and possibly other) variables, a regression curve can be generated and integrated into the algorithm 700, e.g. based on a database of cadaveric studies, to provide optimal screw trajectories in a particular patient at his respective vertebrae whose density and other structural characteristics are known from the 3D spine model 50. By optimizing a particular multi-level plan for spine screw placement (as well as the associated rod(s)) in a particular patient, the algorithm 700 facilitates a treatment plan that is least likely to result real-world biomechanical failure.

In 812, the algorithm 700 may output the final screw trajectories and/or widths and the method may terminate.

From the above description of the invention, those skilled in the art will perceive improvements, changes, and modifications. Such improvements, changes, and modifications within the skill of the art are intended to be covered by the appended claims.

Having described the invention, we claim:

1. A method for optimization of spine screw placement in a spine of a patient, the method comprising:
a) for a first entry point on a surface of a vertebra among a plurality of vertebrae in a spine model representative of the spine of the patient, defining a first plurality of primary rays respectively representing a plurality of screw trajectories for a spine screw within the model entering from the first entry point;
b) eliminating each of the first plurality of primary rays that intersects a boundary of one or more vertebrae of the spine model, representing a surface of an associated vertebra in the patient, thereby establishing a first set of optimized screw trajectories comprising those of the first plurality of primary rays remaining following this step (b);
c) defining, for each of the first set of optimized screw trajectories, a plurality of parallel rays disposed circumferentially around, and extending parallel to, the associated primary ray at a predetermined radius therefrom, and which represent a surface of a spine screw having the screw trajectory represented by the associated primary ray;
d) iteratively adjusting a length of the plurality of parallel rays associated with each of said first set of optimized screw trajectories until an optimized length is determined at which the associated plurality of parallel rays present a maximum-length trajectory for a spine screw that does not intersect any said boundary of the one or more vertebra of the spine model;
e) presenting a list of the first set of optimized screw trajectories and their associated optimized lengths for said first entry point; and
f) implanting a spine screw in a vertebra of the patient corresponding to a selected one of said first set of optimized trajectories.

2. The method of claim 1, further comprising:
g) for a second entry point on a surface of a vertebra among the plurality of vertebrae in said spine model, defining a second plurality of primary rays respectively representing a plurality of screw trajectories for a spine screw within the model entering from the second entry point;
h) eliminating each of the second plurality of primary rays that intersects a said boundary of one or more vertebrae of the spine model, thereby establishing a second set of optimized screw trajectories comprising those of the second plurality of primary rays remaining following this step (h);
i) defining, for each of the second set of optimized screw trajectories, a second plurality of parallel rays disposed circumferentially around, and extending parallel to, the associated primary ray at a predetermined radius therefrom, and which represent a surface of a spine screw having the screw trajectory represented by the associated primary ray;
j) iteratively adjusting a length of the second plurality of parallel rays associated with each of said second set of optimized screw trajectories until an optimized length is determined at which the associated second plurality of parallel rays present a maximum-length trajectory for a spine screw that does not intersect any said boundary of the one or more vertebra of the spine model; and
k) presenting a list of the second set of optimized screw trajectories and their associated optimized lengths for said second entry point; and
l) implanting a spine screw in a vertebra of the patient corresponding to a selected one of said second set of optimized trajectories.

3. The method of claim 2, said first and second entry points being disposed on a surface of the same vertebra of said plurality of vertebrae.

4. The method of claim 3, wherein a location of the first entry point is restrained to be within a predetermined distance of the second entry point.

5. The method of claim 2, said first and second entry points being disposed on respective surfaces of different vertebrae of said plurality of vertebrae.

6. The method of claim 5, wherein a location of the first entry point is restrained to be within a predetermined distance of the second entry point.

7. The method of claim 2, the spine model including mapping of density of the plurality of vertebrae,
wherein the list of the first set of optimized screw trajectories also includes for each optimized screw trajectory thereof a respective first summation of the density of the associated vertebra surrounding or encompassed by the associated plurality of parallel rays, and
wherein the list of the second set of optimized screw trajectories also includes for each optimized screw trajectory thereof a respective second summation of the density of the associated vertebra surrounding or encompassed by the associated plurality of parallel rays;
the method further comprising:
m) calculating a first respective fixation for each optimized screw trajectory in each of the first and second sets of optimized screw trajectories based on the first or second density summation associated therewith;
n) iteratively selecting pairs of the first and second sets of optimized screw trajectories, one from each said set, and calculating an overall fixation for each such pair based on the first respective fixation thereof; and
o) presenting a list of said overall fixation and their associated pairs of the first and second sets of optimized screw trajectories.

8. The method of claim 7, wherein the first respective fixation calculated for each of the first and second sets of optimized screw trajectories is based on a user selected fixation device.

9. The method of claim 7, further comprising:
p) calculating a second respective fixation for each of the first and second sets of optimized screw trajectories based on the respective first or second density summation and an alternative fixation device.

10. The method of claim 9, said first and second entry points being disposed on a surface of the same vertebra of said plurality of vertebrae and said alternative fixation device includes a cross-link connecting a first spline screw in the first entry point to a second spline screw in the second entry point.

11. The method of claim 1, wherein the spine model is derived via a 3-dimensional or volumetric imaging methodology of the patient's spine.

12. The method of claim 1, wherein the spine model includes a mapping of density of the plurality of vertebrae.

13. The method of claim 12, wherein the list of the first set of optimized screw trajectories also includes for each optimized screw trajectory thereof a respective first summation of the density of the associated vertebra encompassed by the associated plurality of parallel rays.

14. The method of claim 12, wherein the list of the first set of optimized screw trajectories also includes for each optimized screw trajectory thereof a respective first summation of the density of the associated vertebra surrounding the associated plurality of parallel rays.

15. The method of claim 2, the model of the vertebrae including a mapping of density of the plurality of vertebrae,
wherein the list of the first set of optimized screw trajectories also includes for each optimized screw trajectory thereof a respective first summation of the density of the associated vertebra surrounding or encompassed by the associated plurality of parallel rays, and
wherein the list of the second set of optimized screw trajectories also includes for each optimized screw trajectory thereof a respective second summation of the density of the associated vertebra surrounding or encompassed by the associated plurality of parallel rays;
the method further comprising:
l) calculating a respective pull-out strength for each optimized screw trajectory in each of the first and second sets of optimized screw trajectories based on the first or second density summation associated therewith; and
m) presenting a list of said pull-out strengths and their associated pairs of the first and second sets of optimized screw trajectories.

16. The method of claim 15, wherein said pull-out strengths less than a user predetermined value are removed from said list of said pull-out strengths.

17. A method for optimization of spine screw placement in a spine of a patient, the method comprising:
a) for a first entry point on a surface of a vertebra among a plurality of vertebrae in a spine model representative of the spine of the patient, defining a first plurality of primary rays respectively representing a plurality of screw trajectories for a spine screw within the model entering from the first entry point;
b) eliminating each of the first plurality of primary rays that intersects a boundary of one or more vertebrae of the spine model, representing a surface of an associated vertebra in the patient, thereby establishing a first set of optimized screw trajectories comprising those of the first plurality of primary rays remaining following this step (b);
c) defining, for each of the first set of optimized screw trajectories, a plurality of parallel rays disposed circumferentially around, and extending parallel to, the associated primary ray at a predetermined radius therefrom, and which represent a surface of a spine screw having the screw trajectory represented by the associated primary ray;
d) iteratively adjusting a length of the plurality of parallel rays associated with each of said first set of optimized screw trajectories until an optimized length is determined at which the associated plurality of parallel rays present a maximum-length trajectory for a spine screw that does not intersect any said boundary of the one or more vertebra of the spine model;
e) calculating a respective first summation of the density of the associated vertebra surrounding or encompassed by the associated plurality of parallel rays based on a mapping of density of the plurality of vertebrae;
f) calculating a respective pull-out strength for each optimized screw trajectory in each of the first set of optimized screw trajectories based on the first density summation associated therewith;
g) presenting a list of the first set of optimized screw trajectories and their associated optimized lengths for said first entry point and said pull-out strengths; and
h) implanting a spine screw in a vertebra of the patient corresponding to a selected one of said first set of optimized trajectories.

18. The method of claim 17, wherein the spine model is derived via a 3-dimensional or volumetric imaging methodology of the patient's spine.

19. The method of claim 17, wherein said pull-out strengths less than a user predetermined value are removed from said list of said pull-out strengths.

20. A non-transitory computer-readable medium having instructions stored thereon that, when executed by a computer, cause the computer to perform a method for optimization of spine screw placement in a spine of a patient, the method comprising:
a) for a first entry point on a surface of a vertebra among a plurality of vertebrae in a spine model representative of the spine of the patient, defining a first plurality of primary rays respectively representing a plurality of screw trajectories for a spine screw within the model entering from the first entry point;
b) eliminating each of the first plurality of primary rays that intersects a boundary of one or more vertebrae of the spine model, representing a surface of an associated vertebra in the patient, thereby establishing a first set of optimized screw trajectories comprising those of the first plurality of primary rays remaining following this step (b);
c) defining, for each of the first set of optimized screw trajectories, a plurality of parallel rays disposed circumferentially around, and extending parallel to, the associated primary ray at a predetermined radius therefrom, and which represent a surface of a spine screw having the screw trajectory represented by the associated primary ray;
d) iteratively adjusting a length of the plurality of parallel rays associated with each of said first set of optimized screw trajectories until an optimized length is determined at which the associated plurality of parallel rays present a maximum-length trajectory for a spine screw that does not intersect any said boundary of the one or more vertebra of the spine model; and
e) presenting a list of the first set of optimized screw trajectories and their associated optimized lengths for said first entry point.
